(19) United States
(12) Patent Application Publication
(10) Pub. No.: US 2012/0136262 A1
(43) Pub. Date: May 31, 2012

(54) ELECTRONIC SPHYGMOMANOMETER AND A METHOD FOR BLOOD PRESSURE MEASUREMENT BY USING AN ELECTRONIC SPHYGMOMANOMETER

(75) Inventors: Yukiya Sawanoi, Nara-shi (JP); Toshihiko Ogura, Kyoto-shi (JP)

(73) Assignee: OMRON HEALTHCARE CO., Ltd., Kyoto (JP)

(21) Appl. No.: 13/307,894

(22) Filed: Nov. 30, 2011

(30) Foreign Application Priority Data
Nov. 30, 2010 (JP) 2010-266519

(51) Int. Cl. A61B 5/0225

(52) U.S. Cl. 600/493

(57) ABSTRACT

An electronic sphygmomanometer includes a cuff containing an air bladder, an air charger, an air discharger, a pressure sensor, a memory; a display, and CPU for executing programs stored in the memory and calculating a blood pressure value based on the pressure changes within the air bladder. The CPU further includes an estimation processing part for calculating an estimated blood pressure value based on the pressure change within the air bladder during the inflation of the air bladder at a first rate, a measurement processing part for measuring a blood pressure value based on the pressure change within the air bladder during the deflation of the air bladder at a second rate, and a determination processing part for comparing the estimated blood pressure value with the reference blood pressure value and determining whether the estimated blood pressure value is within a predetermined range from the reference blood pressure value.

### BACKGROUND OF INVENTION

The present invention relates to an electronic sphygmomanometer having a quick blood pressure checking function and a method of monitoring blood pressure using the electronic sphygmomanometer, and particularly relates to an electronic sphygmomanometer that monitors by measuring blood pressure using a cuff containing an air bladder and a method of monitoring blood pressure measurements with the electronic sphygmomanometer.

Blood pressure is one indicator for analyzing circulatory diseases, and performing risk analysis based on blood pressure is effective at preventing diseases of the cardiovascular system such as strokes, heart failure, heart attacks, and the like.

These diseases have conventionally been diagnosed by blood pressure (casual blood pressure) measured by a medical care provider during a patient visit or during a health checkup. However, through recent research, it is becoming clear that blood pressure measured at home (home blood pressure) is more useful to the diagnosis of circulatory diseases than casual blood pressure. In conjunction with this, sphygmomanometers that can be used at home are growing in popularity.

Many of the sphygmomanometers for home use have adopted blood pressure measurement methods using the Oscillometric method or the auscultatory method. Blood pressure measurement by the Oscillometric method to obtain a blood pressure value by the deflation process wraps a cuff around the measurement area such as the upper arm, and pressure is increased inside the cuff (cuff pressure) to a prescribed pressure (for example 30 mmHg) higher than the systolic pressure, and thereafter, the cuff pressure is gradually or incrementally reduced. The volume change of an artery during the deflation process is measured as a pressure change (amplitude of a pressure pulse wave) superimposed on the cuff pressure, and systolic pressure and diastolic pressure are determined from the change in this amplitude of a pressure pulse wave. Note, measurement of blood pressure by the Oscillometric method is also possible by measuring the amplitude of a pressure pulse wave while increasing cuff pressure.

Meanwhile, blood pressure measurement by the auscultatory method to obtain a blood pressure value by the deflation process, in a similar manner to the Oscillometric method, wraps a cuff around the measurement area such as the upper arm, and cuff pressure is increased to a prescribed pressure higher than the systolic pressure. Thereafter, the Korotkoff sounds generated by the artery during the process of gradually releasing cuff pressure are detected by a microphone provided within the cuff, and the cuff pressure at which the Korotkoff sounds are generated determines the systolic pressure while the cuff pressure at which the Korotkoff sounds weaken or disappear determines the diastolic pressure.

Blood pressure measurement by the Oscillometric method calculates blood pressure according to the change point in the amplitude of a pressure pulse wave as described above, and therefore, the information for a plurality of pressure pulse wave amplitudes are required during the time the cuff pressure changes from at least the systolic pressure to at most the diastolic pressure. Further, measurement accuracy improves with the greater the number of pressure pulse wave amplitudes. In addition, improving measurement accuracy with blood pressure measurement using the auscultatory method requires that the speed at which the cuff pressure is reduced be sufficiently slow. In other words, securing highly precise measurements with either method requires slowing the speed of change in cuff pressure, and therefore the measurement time is longer.

Additionally, in both methods as described above, a blood pressure value is obtained by the deflation process by temporarily increasing the pressure in the cuff a prescribed amount above the systolic pressure and then gradually reducing the pressure, and thus, the total measurement time is lengthened according to how high a person's blood pressure is.

A method for shortening the measurement time has been disclosed in, for example, Japanese Unexamined Patent Application No. 2001-70263 (Patent Document 1), which is a method that estimates the blood pressure value and the pulse rate of the person being measured by the Oscillometric method while pressure is added to the cuff and then calculates and controls the optimal deflation rate for the person being measured according to the estimated blood pressure value and pulse rate. The disclosure of which is incorporated herein by reference. However, the measurement time with this method requires roughly at least 40 seconds, and the need to increase the pressure by a prescribed amount higher than the systolic pressure is not resolved.

In addition, adding pressure to the cuff is required to be done in a short time (for example, at most 10 seconds) in order to prevent generating errors in the measurement value due to homeostasis on the peripheral side of the measurement area. Therefore, measurement accuracy of the blood pressure value and the pulse rate measured during inflation has not been sufficient to enable use in blood pressure monitoring.

Techniques that measure blood pressure by the process of increasing cuff pressure have also been developed for the Oscillometric method. In this case, the cuff inflating speed must be set to be slow (for example 5 mmHg per second or the like) so as to enable sufficient pressure pulse wave amplitude information to be sufficiently secured for measurement regardless of the person's blood pressure value or pulse rate.

As the measurement time becomes longer, the measuring person may begin to feel annoyed at daily blood pressure measuring, which can become a primary factor in killing the desire to continue to measure especially for measuring blood pressure in the home.

One or more embodiments of the present invention facilitate continuous blood pressure monitoring in daily life by providing an electronic sphygmomanometer that can perform a measurement to obtain a blood pressure value by a deflation process using a cuff containing an air bladder so that a user can understand their own blood pressure condition by a simple method and without taking time.

### SUMMARY OF INVENTION

According to one or more embodiments of the invention, an electronic sphygmomanometer comprises: a cuff containing an air bladder for wrapping around a measurement area of a person being examined; an air charger for inflating the air bladder, an air discharger for deflating the air bladder, a sensor for detecting changes of pressure within the air bladder during the inflation and/or deflation of the air bladder;

a memory for storing control programs, a reference blood pressure value, and a result of measurement of blood pressure; a display for displaying a result of measurement of blood pressure, a central processing unit for executing programs stored in the memory and calculating to obtain blood pressure value of the person being examined based on the changes of pressure within the air bladder, the central processing unit further comprising: an estimation processing part for calculating an estimated blood pressure value of the person being examined based on the pressure change within the air bladder during the inflation of the air bladder at a first rate; and a measurement processing part for measuring a blood pressure value of the person being examined based on the pressure change within the air bladder during the deflation of the air bladder at a second rate; and a determination processing part for comparing the estimated blood pressure value with the reference blood pressure value and determining whether the estimated blood pressure value is within a predetermined range from the reference blood pressure value. If the estimated blood pressure value is determined to be within a predetermined range from the reference blood pressure value, measurement operation of the sphygmomanometer is ended without measuring blood pressure by the measurement processing part, and if the estimated blood pressure value is determined to be not within a predetermined range from the reference blood pressure value, blood pressure of the person being examined is measured by the measurement processing part.

According to one or more embodiments of the invention, an electronic sphygmomanometer comprises: a cuff containing an air bladder for wrapping around a measurement area of a person being examined; a pump for charging air to inflate the air bladder, a valve for releasing air to deflate the air bladder, a sensor for detecting changes of pressure within the air bladder during charging and discharging air to and from the air bladder; a memory for storing control programs, a reference blood pressure value, and a result of measurement of blood pressure; a display unit for displaying measured blood pressure, CPU for executing programs stored in the memory and calculating blood pressure of the person being examined based on the changes of pressure within the air bladder, the CPU further comprising: an estimation processing part for calculating an estimated blood pressure value of the person being examined based on the pressure change within the air bladder during the inflation of the air bladder at a first rate; and a measurement processing part for measuring a blood pressure value of the person being examined based on the pressure change within the air bladder during the deflation of the air bladder at a second rate; and a determination processing part for comparing the estimated blood pressure value obtained by the estimation processing with the reference blood pressure value and determining whether the estimated blood pressure value is within a predetermined range from the referenced blood pressure value. If the estimated blood pressure value is determined to be within a predetermined range from the reference blood pressure value, the display unit displays the estimated blood pressure value and measurement operation of the sphygmomanometer is ended.

According to one or more embodiments of the invention, an electronic sphygmomanometer comprises a cuff containing an air bladder for wrapping around a measurement area of a person being examined; means for inflating the air bladder, means for deflating the air bladder, means for detecting changes of pressure within the air bladder during the inflating and/or deflating the air bladder; means for storing control programs, a reference blood pressure value, and a result of measurement of blood pressure; means for displaying measured blood pressure of the person being examined; and means for executing programs stored in the memory and calculating blood pressure of the person being examined based on the changes of pressure within the air bladder, the means for calculating blood pressure further comprising: means for calculating an estimated blood pressure value of the person being examined based on the pressure change within the air bladder during the inflation at a first rate; and means for measuring a blood pressure value of the person being examined based on the pressure change within the air bladder during the deflation at a second rate; and means for comparing the estimated blood pressure value obtained by the estimation processing with the reference blood pressure value. If the estimated blood pressure value is determined to be within a predetermined range from the reference blood pressure value, measurement operation of the sphygmomanometer is ended without performing measurement by the measurement processing part, and if the estimated blood pressure value is determined to be not within a predetermined range from the reference blood pressure value, blood pressure of the person being examined is measured by the measurement processing part.

According to one or more embodiments of the invention, a method of monitoring blood pressure by using an electronic sphygmomanometer comprises wrapping a cuff around a measurement area of a person being examined, inflating an air bladder at a first rate of change; calculating an estimated blood pressure value of the person being examined based on the pressure change of the air bladder during the inflation of the air bladder; comparing the estimated blood pressure value with a reference value and determining whether the estimated blood pressure value is within a predetermined range from the referenced blood pressure value. If the estimated blood pressure value is determined to be not within a predetermined range from the reference value, the air bladder is inflated to a predetermined pressure level at a first rate and the air bladder is deflated to a predetermined pressure level at a second rate, and the change of blood pressure value is measured during the deflation of the air bladder, and if the estimated blood pressure value measured is determined to be within a predetermined range from the reference blood pressure value, the cuff is deflated without measuring blood pressure.

### BRIEF DESCRIPTION OF DRAWINGS

FIG. 1 is a block diagram illustrating a detailed example of a configuration of an electronic sphygmomanometer (herein after abbreviated as sphygmomanometer) that relates to the first embodiment.

FIG. 2 is a block diagram illustrating a detailed example of a functional configuration of a sphygmomanometer that relates to the first embodiment.

FIG. 3 is a flowchart indicating the measurement operation in the sphygmomanometer that relates to the first embodiment.

FIG. 4 is a flowchart indicating the measurement operation in the sphygmomanometer that relates to the first embodiment.

FIG. 5 is a diagram illustrating a detailed example of a display at ST6 of FIG. 3.

FIG. 6 is a diagram illustrating a detailed example of a display at ST12 of FIG. 4.

FIG. 7 is a diagram illustrating a detailed example of a display at ST8 of FIG. 3.

FIG. 8 is a diagram illustrating a detailed example of information stored in memory.

FIG. 9 is a block diagram illustrating a detailed example of a functional configuration of a sphygmomanometer that relates to the second embodiment.

FIG. 10 is a flowchart indicating the first example of the measurement operation in the sphygmomanometer that relates to the second embodiment.

FIG. 11 is a diagram illustrating a detailed example of a display at ST6 in the first operating example.

FIG. 12 is a flowchart indicating an example of an operation that corresponds to FIG. 3 from among measurement operations in the sphygmomanometer that relates to the second embodiment.

FIG. 13 is a diagram illustrating a detailed example of a display at ST6 in the second operating example of the second embodiment.

FIG. 14 is a flowchart indicating a third example of an operation that corresponds to FIG. 3 from among measurement operations in the sphygmomanometer that relates to the second embodiment.

FIG. 15 is a diagram illustrating a detailed example of a display at ST6 in the third operating example of the second embodiment.

FIG. 16 is a flowchart indicating an operating example of the sphygmomanometer that relates to the third embodiment.

### DETAILED DESCRIPTION

Hereinafter, embodiments of the present invention will be described with reference to the drawings. In the following description, the same reference numbers will be attached with the same components and compositional elements. The names and functions of these are also the same. In embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid obscuring the invention.

### First Embodiment

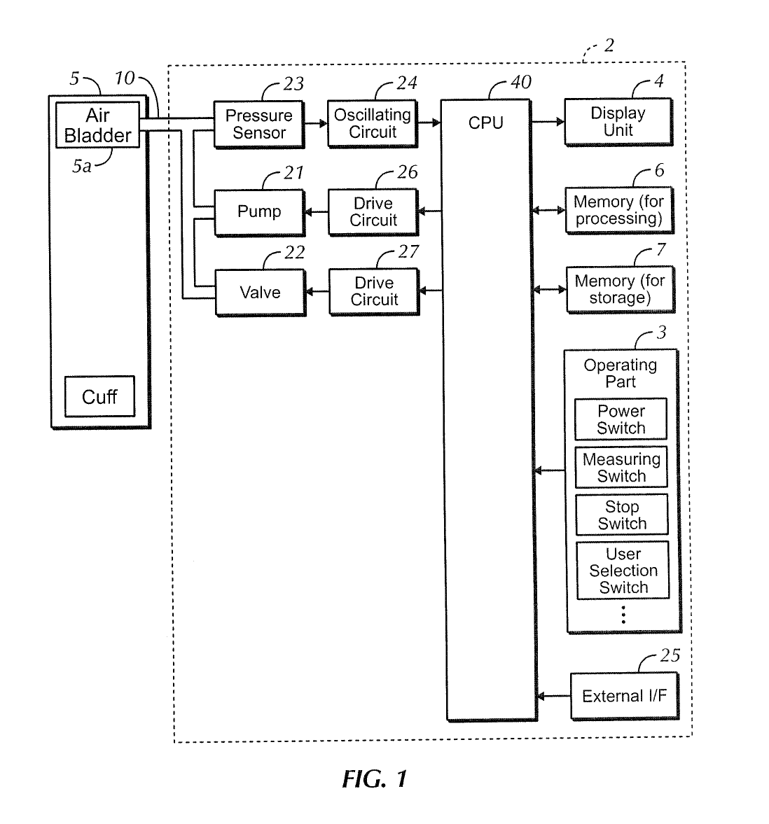

FIG. 1 is a block diagram illustrating a detailed example of one configuration of an electronic sphygmomanometer (herein after abbreviated as sphygmomanometer) that relates to the first embodiment of the present invention.

In referencing FIG. 1, the sphygmomanometer includes a cuff 5 which is a measuring band for wrapping around a measurement area such as the upper arm, a main body part 2, and an air tube 10 is connected to these. A display unit 4 and an operating part 3 are arranged on the face of the main body part 2.

The operating part 3 includes a plurality of switches such as a power switch for instructing the power to turn On/Off, a measuring switch for instructing the measurement operation to begin, a stop switch for instructing the measurement to stop, a user selection switch for selecting the person to be measured, and the like.

An air bladder 5A connected to an air tube 10 is included with the cuff 5. The main body part 2 of the sphygmomanometer 1 includes the air bladder 5A, a pump 21, a valve 22, a pressure sensor 23 connected by an air tube 10, a central processing unit (CPU) 40 for controlling the sphygmomanometer 1 overall, memory 6 for processing, memory 7 for storage, and an external interface (I/F) 25, which is an interface for exchanging data by connecting with external devices not shown. The pressure sensor 23 is connected to an oscillating circuit 24, the pump 21 is connected to a drive circuit 26, and the valve 22 is connected to a drive circuit 27. The oscillating circuit 24, the drive circuit 26, and the drive circuit 27 are electrically connected to the CPU 40. The display unit 4 and the operating part 3 are further connected to the CPU 40.

The pressure sensor 23 is a capacitance type pressure sensor in which the capacitance value changes depending on the change in pressure within the air bladder 5A.

The oscillating circuit 24 is electrically connected to the CPU 40, and inputs a signal with an oscillating frequency according to the capacitance value of the pressure sensor 23 input into the CPU 40.

The memory 6 for processing stores a control program or the like executed by the CPU 40. Furthermore, the memory 6 for processing can also act as a work area for when the CPU 40 executes a program.

The CPU 40 executes a prescribed program stored in the memory 6 based on the operating signal input from the operating part 3 and outputs control signals to drive circuit 26 and drive circuit 27. The drive circuit 26 and drive circuit 27 drive the pump 21 and the valve 22 according to the control signal.

The pump 21 is controlled by the drive circuit 26 according to the control signal from the CPU 40 and injects air into the air bladder 5A. The opening and closing of the valve 22 is controlled by the drive circuit 27 according to the control signal from the CPU 40 and emits air from within the air bladder 5A.

The CPU 40 executes a prescribed process based on the pressure change within the air bladder 5A obtained from the pressure sensor 23, and according to the result thereof, outputs the control signals described above to the drive circuit 26 and to the drive circuit 27. Further, the CPU 40 calculates the blood pressure value based on the pressure change within the air bladder 5A obtained from the pressure sensor 23, performs a process to display the measured result on the display unit 4, and outputs the data to be displayed and a control signal to the display unit 4. Furthermore, the CPU 40 performs a process for storing the blood pressure value into the memory 7.

When measuring the blood pressure using the sphygmomanometer 1, the cuff 5 is wrapped around the measurement area such as the upper arm, and the power switch and the user selection switch are pressed in that order, and then pressing the measuring switch starts the measuring action operation.

The measurement operation of the sphygmomanometer 1 is divided into an inflation process and a deflation process. With the inflation process, the air bladder 5A is pressurized by the pump 21 at a prescribed inflating speed until achieving a prescribed pressure that is higher than the systolic pressure of the person being measured. With the deflation process, the pressure within the air bladder 5A is gradually released by the valve 22 at a prescribed pressure releasing speed, which is a lower change rate than the change rate of the internal pressure of the inflating speed from the prescribed pressure.

The CPU 40 of the sphygmomanometer 1 measures the blood pressure value and/or the pulse rate of the person being measured based on the pressure change superimposed on the change in internal pressure by the inflating speed in the inflating process. Because the inflation of the cuff must be done in a short period of time, the accuracy of the blood pressure value and the pulse rate measured during inflation is insufficient to be used to measure accurate blood pressure. However, it is possible to estimate the blood pressure value of the person being measured with a certain level of accuracy from the low accuracy blood pressure value that was obtained by a measurement in a short period of time. Although a blood pressure value of the person being measured obtained by the measurement in the inflating process is used as the estimated blood pressure value in the present embodiment, a value, for example 10 mmHg, to be added to or reduced from the blood pressure value measured by the inflation process can be made to be the estimated value in the event that the blood pressure of the user is usually higher or lower. Adjusting the estimated value based on the blood pressure trend of the person being measured is effective in securing safety in blood pressure monitoring of the user. The estimated value obtained in this manner is stored in the memory 7 for storage. Further, the CPU 40 calculates the blood pressure value and/or the pulse rate of the person being measured based on the pressure change superimposed on the change in internal pressure by the deflation speed in the deflation process. Note, in the description to be given hereafter, the blood pressure value will be an estimated measurement and calculated value. The pulse rate can also be treated in a similar manner.

Further, the CPU 40 of the sphygmomanometer 1 determines whether the estimated measurement value in the inflation process is within a predetermined range (hereinafter abbreviated as the reference range). Further, when outside the reference range, it proceeds to the step for processing the calculation of the blood pressure value in the deflation process. Meanwhile, when within the reference range, the air within the air bladder 5A is discharged after the inflation process without performing the process for the deflation process and the measurement operation ends.

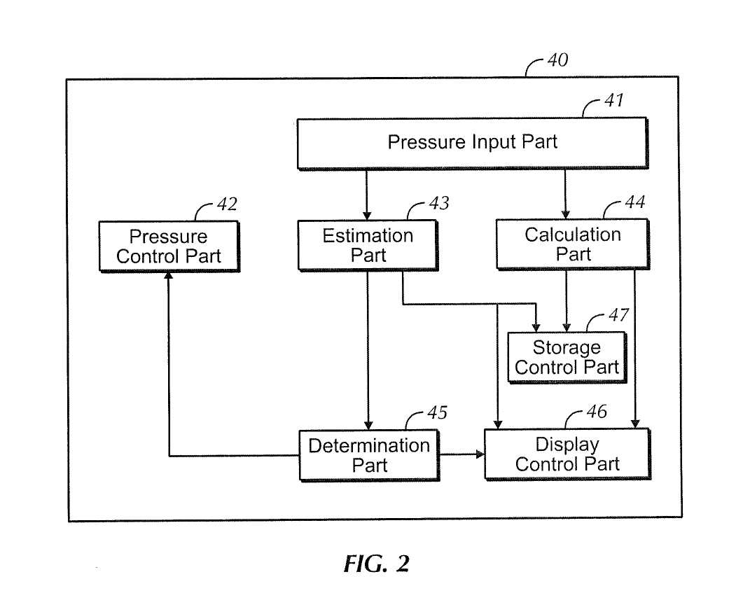

FIG. 2 is a block diagram illustrating a detailed example of a functional configuration for performing the above operation with the sphygmomanometer 1. Each function in FIG. 2 is a function formed primarily by the CPU 40 whereby the CPU 40 reads and executes a program stored in the memory 6. Note, at least a portion thereof may also be executed by the hardware configuration illustrated in FIG. 1.

In reference to FIG. 2, the CPU 40 includes a pressure input part 41 for obtaining the pressure within the air bladder 5A and receiving input of a sensor signal from the pressure sensor 23, an internal pressure control part 42 for controlling the pressure within the air bladder 5A, an estimation part 43 for estimating the blood pressure value from the pressure change within the air bladder 5A in the inflation process, a calculation part 44 for calculating the blood pressure value from the pressure change within the air bladder 5A in the deflation process, a determination part 45 in which the reference range is stored in advance for determining whether the estimated value is within the reference range by comparing the estimated value obtained in the inflating process with a reference range and for determining according to the result thereof whether to execute internal pressure control of the deflation process, a display control part 46 to perform a process that displays the estimated value, blood pressure value, and the determination result by the determination part 45 on the display unit 4, and a storage processing part 47 for storing the estimated value and the blood pressure value in an area of the memory 7 that corresponds to the person being measured.

Here, the reference range can be set to be a range that is below the reference value, which value represents hypertension as a reference value such as 135 mmHg for systolic pressure and 85 mmHg for diastolic pressure as established as a standard for hypertension in home blood pressure by, for example, The Japanese Society of Hypertension. The determination part 45 compares at least one of the estimated values (the blood pressure value of the person being measured estimated based on the blood pressure value measured in the inflating process) of systolic pressure and diastolic pressure with the reference value and determines whether the estimated value is equal to or less than the reference value. Note, in the explanation given below, the estimated value for systolic pressure will be abbreviated as “estimated systolic pressure” and the estimated value for diastolic pressure will be abbreviated as “estimated diastolic pressure.”

Further, the estimation part 43 undertakes a calculation to estimate the “estimated systolic pressure” and the “estimated diastolic pressure” based on the systolic pressure and diastolic pressure of the person being measured that is measured by the change in the pressure within the air bladder 5A during the inflation process, and the determination part 45 may also calculate the average blood pressure (hereinafter, estimated average blood pressure) based on these. The estimated average blood pressure can be calculated by the following Formula 1 from the estimated systolic pressure and the estimated diastolic pressure:

estimated average blood pressure=(estimated systolic pressure−estimated diastolic pressure)/3+estimated diastolic pressure Formula 1.

Or, the determination part 45 can designate the pressure within the air bladder 5A at the point in which the maximum value for the amplitude of a pressure pulse wave obtained in the inflating process is detected, as the estimated average blood pressure.

Furthermore, the determination part 45 may also determine whether the estimated average blood pressure is equal to or less than the reference value by using the value obtained in a similar manner to that in Formula 1 from the blood pressure value established as a standard for hypertension by The Japanese Society of Hypertension described above, as the reference value.

The determination part 45 may also compare these according to a magnitude correlation between the estimated value and the reference value, or it may calculate the difference between the estimated value and the reference value and compare these to see whether they are within the reference range, or it may calculate a ratio between the estimated value and the reference value and compare these to see whether they are within the reference range.

In the explanation given below, the determination part 45 will be that which determines the estimated value by whether it exceeds the reference value based on a magnitude correlation between the estimated value and the reference value. As another example, the determination part 45 may proceed to blood pressure measurement with the deflation process when the difference compared to the blood pressure value stored as a previous measured value for the person being tested exceeds a prescribed value (for example, 10 mmHg).

Further, it is understood that blood pressure always fluctuates, and that fluctuation can fluctuate within one day (same day fluctuation), fluctuate depending on the day, and can fluctuate according to the season. Therefore, when comparing to a stored blood pressure value, it can be configured to compare with a stored value that is closest to one or more of the time of day, the day of the week, or the date and time of the measurement. An example of the closest stored value can be, for example, within plus or minus one hour from the current measuring time if referencing the time of day, or it can be the day of the week if referencing the day of the week, or plus or minus one week from the month and day of the measurement if referencing the season.

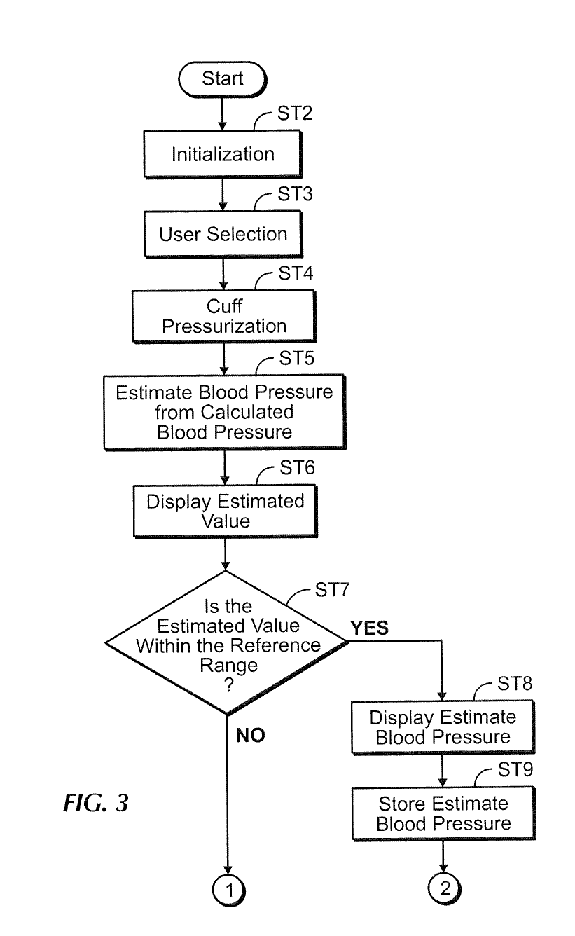

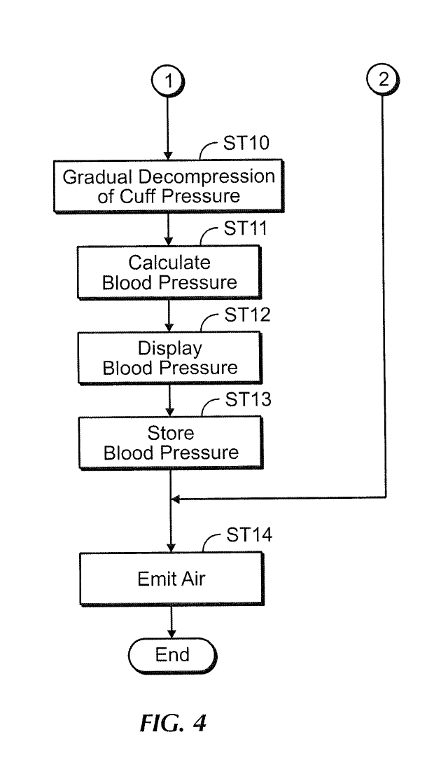

FIG. 3 and FIG. 4 are flowcharts indicating the measurement operation in the sphygmomanometer 1. The operations indicated in the flowcharts in FIG. 3 and FIG. 4 are realized by the CPU 40 reading the program stored in the memory 6 and controlling each of the parts in FIG. 1 to demonstrate each of the functions in FIG. 2. Further, the operations indicated in the flowcharts in FIG. 3 and FIG. 4 are initiated by the CPU 40 receiving the input of the operating signal that indicates that the power switch included in the operating part 3 has been pressed.

To begin with referencing FIG. 3, the measurement operation begins by the pressing of the measurement starting switch. At step (hereinafter abbreviated as ST) 2, the CPU 40 performs a 0 mmHG correction of the pressure sensor 23 and initializes a prescribed area of the memory 6 for processing. Next, the person to be measured is identified by pressing the user selection switch in ST 3. By providing a configuration in which a plurality of measurement switches A, B, C, . . . are provided, user A can press switch A when using the device, and user B can press switch B when using the device, and so forth so that the trouble of inputting a user ID can be omitted and measurement for a specific user can be simply and quickly initiated. Note, a step for a user selection is not required in the sphygmomanometer according to one or more embodiments of the present invention and the user selection switch/step can be omitted when using the sphygmomanometer for a specific person.

Next, in ST 4, the CPU 40 opens the valve 22 and applies a prescribed drive voltage E1 to the drive circuit 26 to run the pump 21. By doing so, the air bladder 5A is gradually pressurized at an inflation rate according to the drive voltage E1.

At ST 5, which is the inflation process, the CPU 40 extracts the vibrational component (pressure pulse wave amplitude) in conjunction with the volume change of the artery that is superimposed on the pressure change within the air bladder 5A and calculates the blood pressure value and the pulse rate by a prescribed calculation method stored in the memory. The estimated value is obtained by the calculated blood pressure value.

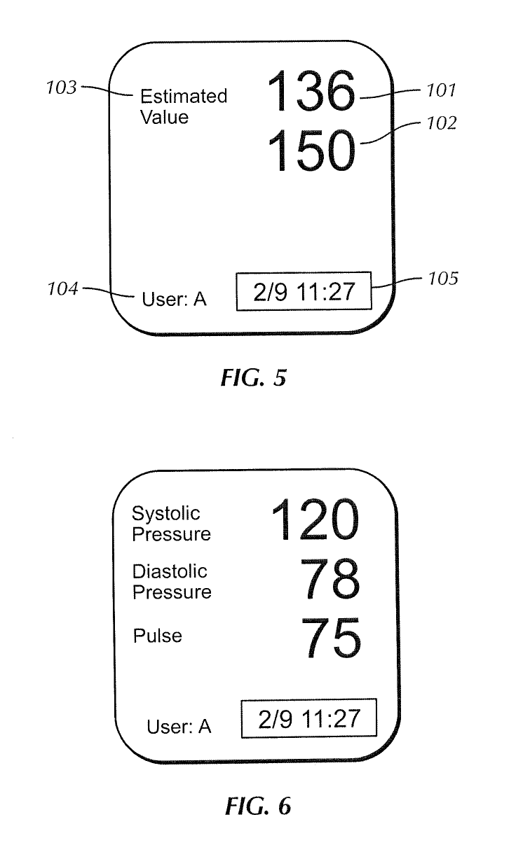

In ST 6, CPU 40 executes a process to display the blood pressure value and pulse rate on the display unit 4. FIG. 5 is a diagram illustrating a detailed example of a display of ST6.

In reference to FIG. 5, the CPU 40 displays the indication 101 of the estimated blood pressure value obtained at ST 6, the indication 102 of the pressure within the air bladder 5A at that moment, the indication 103 that the displayed value is an estimated value, the indication 104 that displays the selected person being measured, and the indication 105 of the current date and time as the measurement date and time on the display unit 4. In other words, the systolic pressure estimated in ST 5 is displayed in ST 6 together with an indication that it is an estimated blood pressure. Further, the current internal pressure is also displayed because the current pressure within the air bladder 5A during blood pressure measurement must be displayed.

By doing so, the user can understand that the displayed value is an estimated value in the inflating process and can use it to determine whether he/she needs to measure his/her accurate blood pressure. Further, the user can understand the current pressure within the air bladder 5A.

Note, although an example is given in FIG. 5 in which the systolic pressure is displayed as the estimated value, either the systolic pressure or pulse rate, or both, can be displayed. When displaying both the estimated blood pressure and the estimated pulse rate, the CPU 40 can display them simultaneously on the display unit 4 or either one or more can be mutually displayed on the display unit 4. In other words, the CPU 40 can be configured to display one item at a time in the order of, for example, the estimated systolic pressure, the estimated diastolic pressure, and the estimated pulse rate.

Further, the CPU 40 compares the estimated value with a reference value described above and determines whether the estimated value is within the reference range. For example, when using the blood pressure value established as a standard for hypertension by The Japanese Society of Hypertension as the reference value, the CPU 40 determines whether the estimated value is within the reference range, which is to say, the CPU 40 determines whether the estimated value is equal to or less than the reference value or if it exceeds the reference value. Furthermore, when it is determined that the estimated value exceeds the reference value (NO in ST 7), the pump 21 continues inflation until the pressure within the air bladder 5A reaches a prescribed pressure that is equal to or greater than the systolic pressure in order to proceed to internal pressure control for the deflation process. Note, the prescribed pressure given here may be a pressure determined in advance. When systolic pressure is estimated in ST 6, a predetermined value (for example 30 mmHg) may be added to the estimated value to calculate the prescribed pressure.

When the pressure within the air bladder 5A reaches a prescribed pressure, in reference to FIG. 4, the CPU 40 at ST 10 stops the pump 21 and applies a prescribed drive voltage E2 from the drive circuit 27 so as to gradually open the valve 22. By doing so, the air bladder 5A is gradually decompressed at a deflation rate according to the drive voltage E2. According to one or more embodiments of the present invention, the drive voltage E2 given here is preferably established so that the rate of change for the pressure within the air bladder 5A during the deflation process is slower than the rate of change for the pressure within the air bladder 5A during the inflating process in order to increase the accuracy of the blood pressure measurement in the deflation process.

At ST 10, which is the deflation process, the CPU 40 extracts the vibrational component in conjunction with the volume change of the artery that is superimposed on the pressure change within the air bladder 5A and calculates the blood pressure value (systolic pressure, diastolic pressure, and pulse rate and the like) according to a prescribed calculation method.

The CPU 40, upon completion of the calculation of the blood pressure value, executes a process at ST 12 to display the blood pressure value calculated in ST 11 on the display unit 4 as a measured result. FIG. 6 is a diagram illustrating a detailed example of a display at ST 12. As illustrated in FIG. 6, the systolic pressure, diastolic pressure, and pulse rate calculated in ST 12 are displayed as the measured results in ST 12.

Further, the CPU 40 executes a process at ST 13 to store the associated date and time of the measurement in the memory 7. At this time, the estimated value obtained in ST 5 may also be jointly stored as will be described hereinafter.

Thereafter, in ST 14, the CPU 40 releases the valve 22. By so doing, the air within the air bladder 5A is discharged. This ends one series of operations.

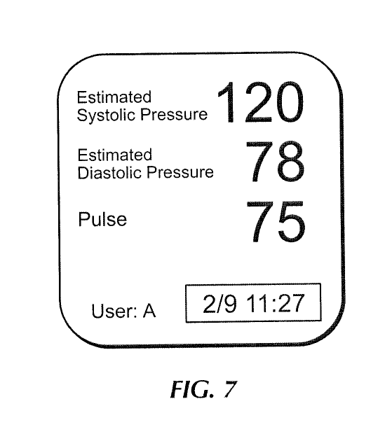

Meanwhile, if it is determined that the estimated value obtained in ST 5 does not exceed the reference value (Yes in ST 7), the CPU 40 in ST 8 executes a process to display the blood pressure value calculated in ST 5 on the display unit 4 as the measured result. FIG. 7 is a diagram illustrating a detailed example of a display at ST 8. As illustrated in FIG. 7, the systolic pressure, diastolic pressure, and pulse rate calculated in ST 5 are displayed as the measured results in ST 8. Note, that these values are calculated in the inflating process and are, in other words, estimated values.

By so doing, the user can know the estimated value obtained by the inflating process as a rough measured value even when the measurement process is not undertaken as will be described hereinafter.

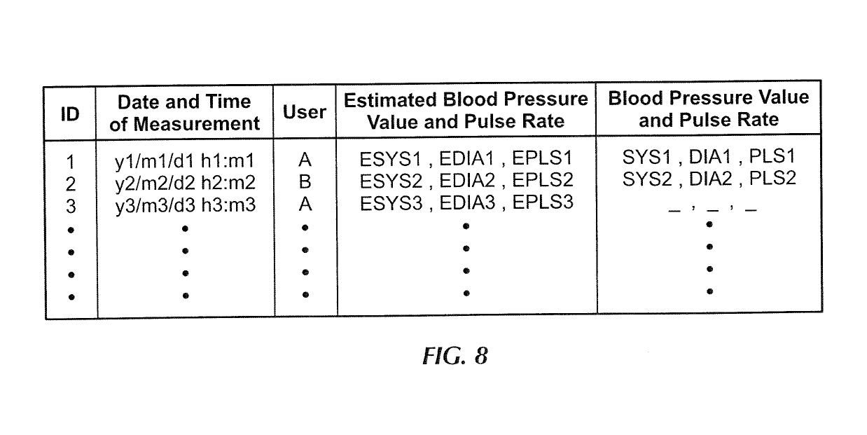

Further, the CPU 40 executes a process at ST 9 to store the associated estimated value and the date and time of the measurement in the memory 7. FIG. 8 is a diagram illustrating a detailed example of information stored in the memory 7.

In reference to FIG. 8, performing the process of ST 9 stores the date and time of the measurement, information specific to the user being measured, and the associated estimated value into the memory 7. Note, although an example in which information identifying a person being measured is associated and stored in the example illustrated in FIG. 8, information in a corresponding area may also be associated and stored when a storage area has been prepared in the memory 7 and associated with a person being measured.

Note, in the flowcharts illustrated in FIG. 3 and FIG. 4, an example is given in which the estimated value in ST 9 is stored in the memory 7 only when it is determined that the estimated value does not exceed the reference value. However, as illustrated in FIG. 8, the measurement operation can be performed in the deflation process following ST 10 described above and can also be assigned to a measured value obtained by such operation and stored.

After completing the process of ST 8 and ST 9, the CPU 40 proceeds to the process in ST 14. In other words, the pump 21 is stopped and the valve 22 is released without performing the operation for blood pressure measurement in ST 10 and ST 11. By doing so, the air within the air bladder 5A is discharged without performing the operation for the deflation process as described above after the inflating process. This ends one series of operations.

By performing the measurement operation as described above with the sphygmomanometer 1, the measurement process can be completed with only the operation for the inflating process when the estimated value in the inflating process does not exceed the reference value. Therefore, the time required for the entire measurement operation in this case can be significantly shortened. Furthermore, in this case, a value can be obtained that was calculated as a measurement result faster than when performing the normal measurement operation.

### Second Embodiment

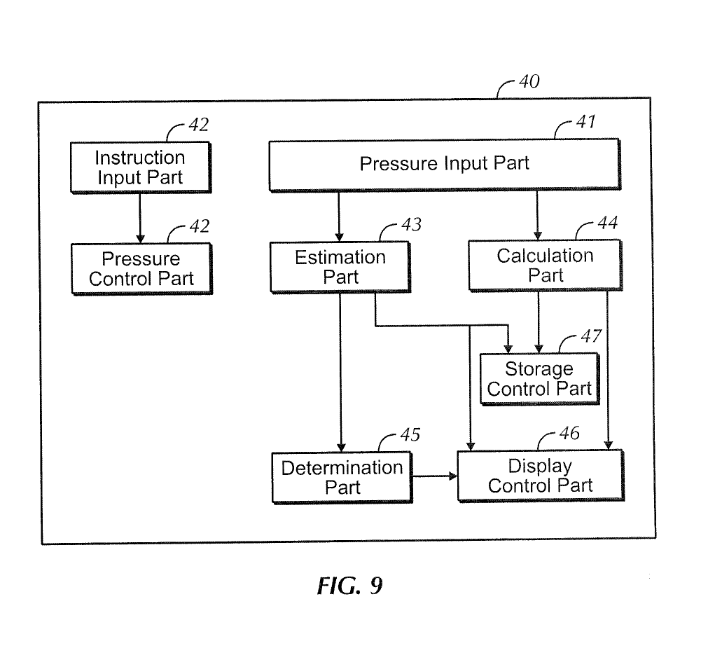

FIG. 9 is a block diagram illustrating a detailed example of a functional configuration of a sphygmomanometer 1 that relates to the second embodiment. In referencing FIG. 9, the CPU 40 in the second embodiment, in addition to the functions illustrated in FIG. 2, further includes an instruction input part 48 for receiving input of an operating signal according to an instructed operation by the user.

The display control part 46 performs a process to display on the display unit 4 operation buttons or a guide to the operation buttons according to the determination results by the determination part 45. Further, the instruction input part 48 receives input of an operating signal of an operation button according to a guide displayed on the display unit 4, or an instructed operation of an operation button displayed on the display unit 4. The internal pressure control part 42 performs pressure control within the air bladder 5A based on the operating signal received by the instruction input part 48.

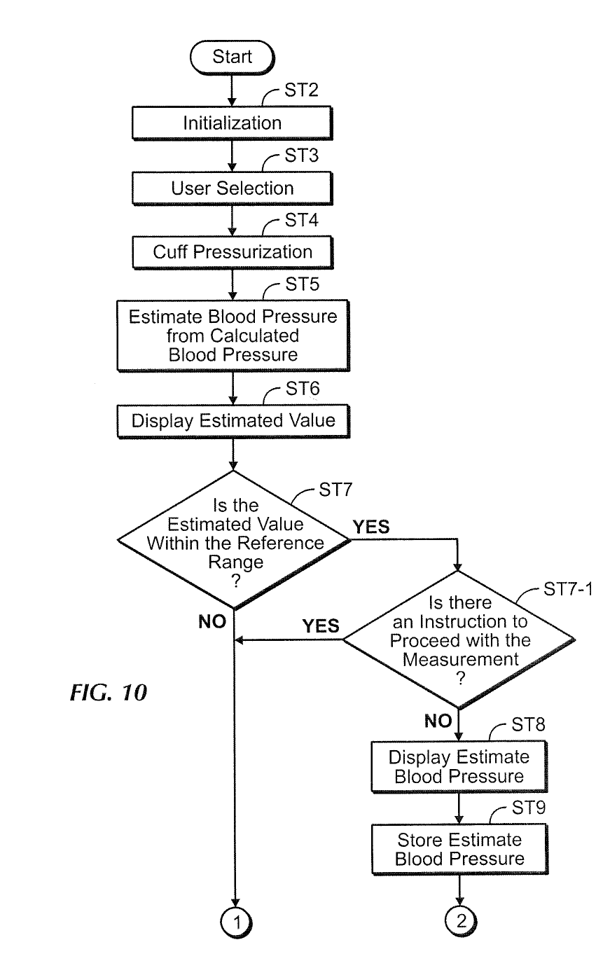

FIG. 10 is a flowchart indicating a first example that corresponds to FIG. 3 from among measurement operations in the sphygmomanometer 1 that relates to the second embodiment.

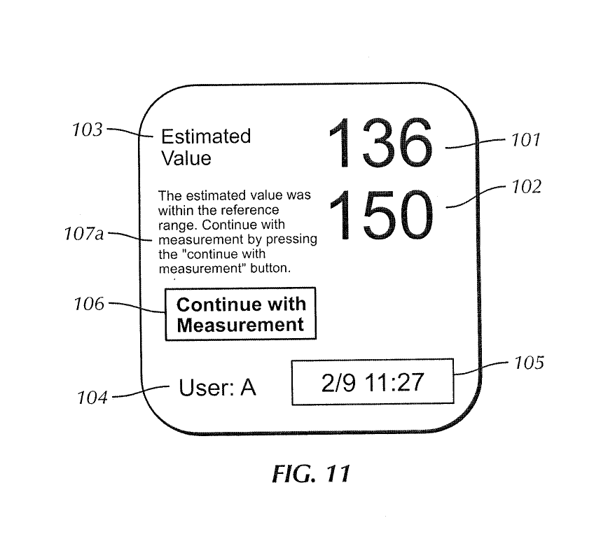

In the first operating example in the second embodiment with reference to FIG. 10, the CPU 40, when displaying the estimated value in ST 6, jointly displays the operation button for instructing the measurement operation to proceed to the operation for the deflation process subsequent to ST 10 as well as the fact that the estimated value obtained in ST 5 does not exceed the reference value. FIG. 11 is a diagram illustrating a detailed example of a display at ST6 in the first operating example of the second embodiment.

In reference to FIG. 11, the CPU 40, in addition to the display illustrated in FIG. 5, further displays the display 106 of the operation button for instructing the measurement operation to proceed to the operation for the deflation process subsequent to ST 11 and the display 107A that indicates that the estimated value does not exceed the reference value. Note, although an example was given in FIG. 11 in which the display 106 of the operation button was done with the display unit 4 as a touch panel, the display 106 may also be a content that guides an operation button used for instructing that the measurement operation is to move to the operation for the deflation process subsequent to ST 10 from among operation buttons that are provided on the operating part 3.

By providing this type of display, the user can understand that the estimated value is within the reference range and that the measurement operation can be omitted while at the same time have the ability to instruct to proceed to the measurement operation in the deflation process.

In the first operating example in the second embodiment, the CPU 40 proceeds to the measurement operation in the deflation process thereafter when receiving the operating signal along the related screen (Yes in ST 7 and Yes in ST 7-1) even when the estimated value obtained in ST 5 does not exceed the reference value. Note, when the estimated value obtained in ST 5 does not exceed the reference value and an operating signal is not received within a predetermined time period (Yes in ST 7 and No in ST 7-1), a process is performed to display the estimated value of ST 17 as the measured result and a process is performed to store the estimated value of ST 18, and this ends one series of operations.

By performing the operation according to the first example, the user can execute the measurement operation for the deflation process by an operation by the user without automatically skipping the measurement operation even if the estimated value is within the reference range and it is possible to omit the measurement operation in the deflation process. Therefore, the user does not lose usability.

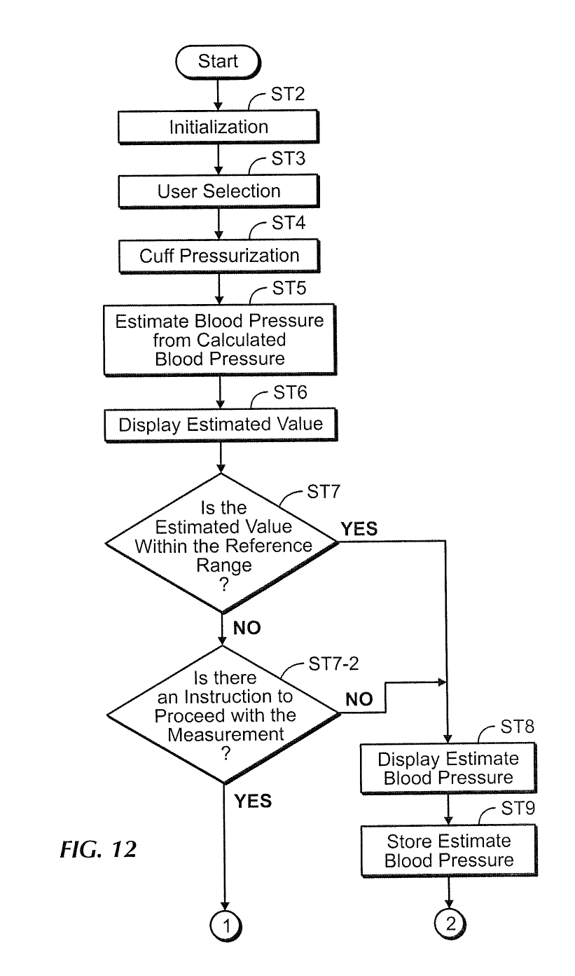

Further, FIG. 12 is a flowchart indicating a second example of an operation that corresponds to FIG. 3 from among measurement operations in the sphygmomanometer 1 that relates to the second embodiment.

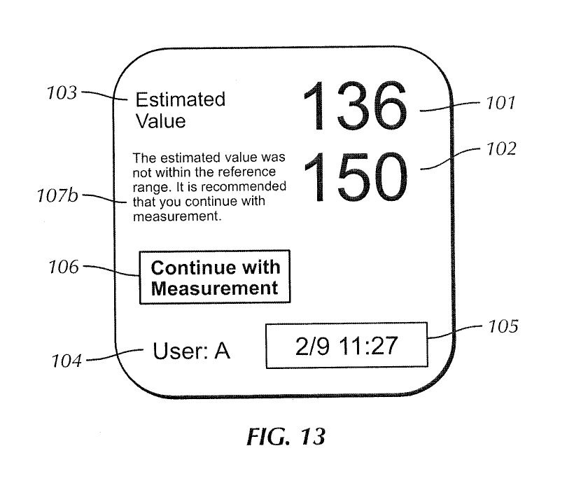

In the second operating example in the second embodiment with reference to FIG. 12, the CPU 40, when displaying the estimated value in ST 6, jointly displays the operation button for instructing the measurement operation to proceed to the operation for the deflation process subsequent to ST 10 as well as the fact that the estimated value obtained in ST 5 exceeds the reference value. FIG. 13 is a diagram illustrating a detailed example of a display at ST6 in the second operating example in the second embodiment.

In reference to FIG. 13, the CPU 40, in addition to the display illustrated in FIG. 5, further displays the display 108 of the operation button for instructing the measurement operation to proceed to the operation for the deflation process subsequent to ST 10 and the display 107B that indicates that the estimated value exceeds the reference value and prompts the measurement operation in the deflation process. Note, although an example was also given in FIG. 13 in which the display 108 of the operation button was done with the display unit 4 as a touch panel, the display 108 may also be a content that guides an operation button used for instructing to move to the operation for the deflation process subsequent to ST 10 from among operation buttons that are provided on the operating part 3.

By providing this type of display, the user can understand the necessity of the measurement operation in the deflation process while at the same time have the ability to instruct to proceed to the measurement operation in the deflation process.

In the second operating example in the second embodiment, the CPU 40 proceeds to the measurement operation in the deflation process thereafter when receiving the operating signal along the related screen (No in ST 7 and Yes in ST 7-2) when the estimated value obtained in ST 5 exceeds the reference value. Note, even when the estimated value obtained in ST 5 exceeds the reference value and an operating signal is not received within a predetermined time period (No in ST 7 and No in ST 7-1), a process is performed to display the estimated value of ST 8 as the measured result and a process is performed to store the estimated value of ST 9, and this ends one series of operations.

By performing the operation according to the second example, the user can execute the measurement operation for the deflation process by an operation by the user with a full understanding as a result of not automatically proceeding to the measurement operation when the estimated value is outside the reference range. Therefore, the user does not lose usability.

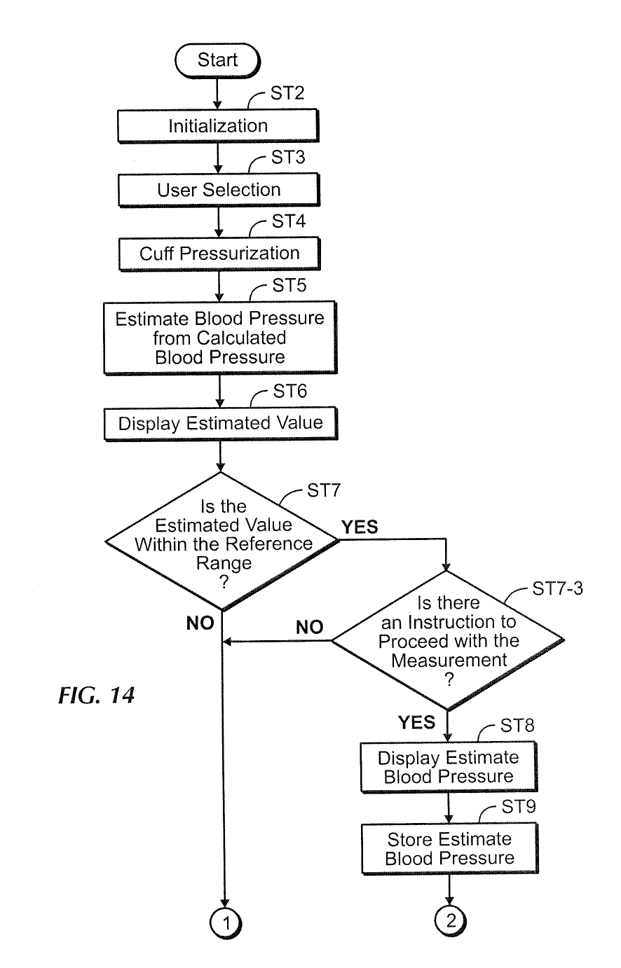

Further, FIG. 14 is a flowchart indicating a third example of an operation that corresponds to FIG. 3 from among measurement operations in the sphygmomanometer 1 that relates to the second embodiment.

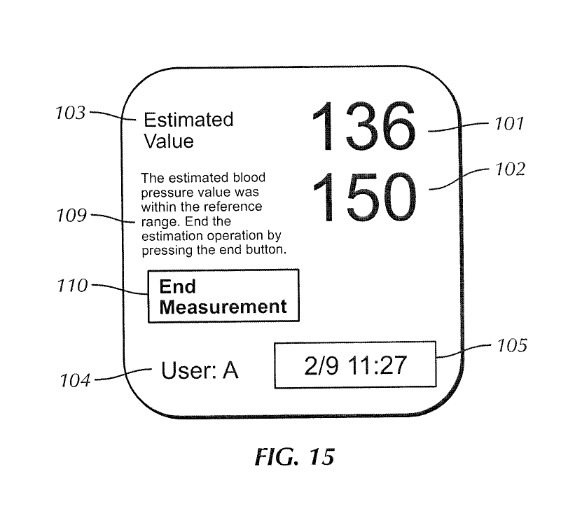

In the third operating example in the second embodiment with reference to FIG. 14, the CPU 40, when displaying the estimated value in ST 6, jointly displays the operation button for instructing the completion of one series of operations without the measurement operation proceeding to the operation for the deflation process subsequent to ST 10 as well as the fact that the estimated value obtained in ST 5 does not exceed the reference value. FIG. 15 is a diagram illustrating a detailed example of a display at ST8 in the third operating example of the second embodiment.

In reference to FIG. 15, the CPU 40, in addition to the display illustrated in FIG. 5, further displays the display 110 of the operation button for instructing the completion of one series of operations without the measurement operation proceeding to the operation for the deflation process subsequent to ST 10 and the display 109 that indicates that the estimated value does not exceed the reference value. Note, although an example was also given in FIG. 15 in which the display 110 of the operation button was done with the display unit 4 as a touch panel, the display 110 may also be a content that guides an operation button used for instructing to end one series of operations without moving to the operation for the deflation process subsequent to ST 10 from among operation buttons that are provided on the operating part 3.

By providing this type of display, the user can understand that the estimated value is within the reference range and that the measurement operation can be omitted while at the same time have the ability to instruct not to proceed to the measurement operation in the deflation process.

In the third operating example in the second embodiment, the CPU 40, when the estimated value obtained in ST 5 does not exceed the reference value and an operating signal is received along the related screen (Yes in ST 7 and Yes in ST 7-3), a process is performed to display the estimated value of ST 17 as the measured result and a process is performed to store the estimated value of ST 18, and this ends one series of operations. Note, it proceeds to the measurement operation in the deflation process thereafter when not receiving the operating signal within a predetermined time period (Yes in ST 7 and No in ST 7-3) even when the estimated value obtained in ST 5 does not exceed the reference value.

By performing the operation according to the third example, the user can omit the measurement operation for the deflation process by an operation by the user without automatically skipping the measurement operation even if the estimated value is within the reference range and it is possible to omit the measurement operation in the deflation process. Therefore, the user does not lose usability.

Note, although an operation is received by displaying an operation button together with the estimated value in the second embodiment, it may also be configured so that an operation is received by displaying an operation button without displaying the comparison results between the estimated value and the reference value when beginning the measurement operation.

### Third Embodiment

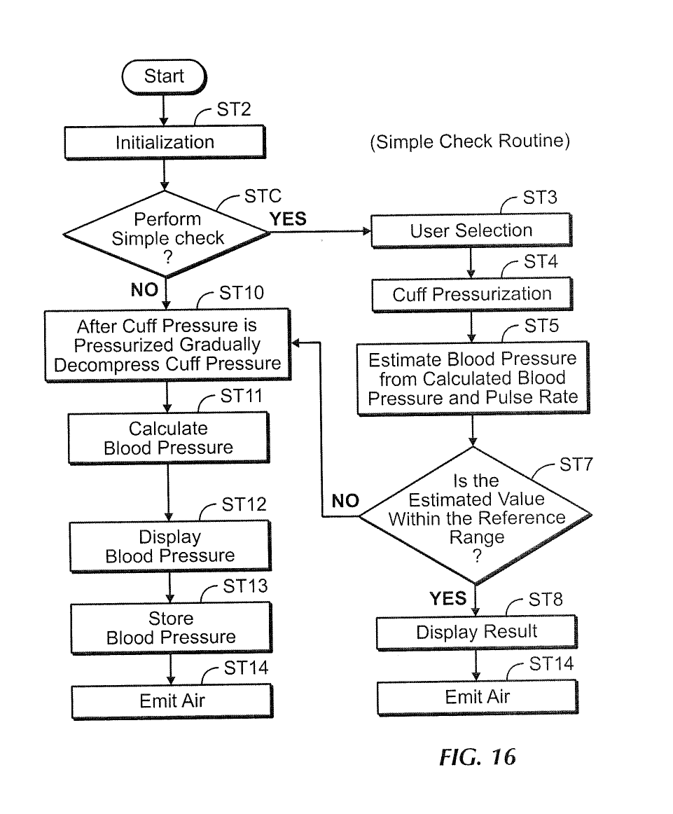

FIG. 16 is a flowchart indicating an example of an operation in the third embodiment from among measurement operations in the sphygmomanometer 1.

In the operating example in the third embodiment with reference to FIG. 16, the user can select in the check step C (STC) whether to perform a simple check of the blood pressure. When the user selects to perform a simple check, it proceeds to the following simple check routine steps. If the user selects not to perform a simple check, the standard blood pressure measurement (measuring blood pressure by a deflation process of a cuff after inflating the cuff) is performed thereafter from step 10 and after.

When a simple check is selected to be performed at STC, the user inputs their ID (ST 3) if they have completed registration with the corresponding sphygmomanometer. When the user's ID is entered, the measured value for the history of the corresponding user stored in the memory 7 for storage is read. If the user has not yet registered, an ID number is entered for a new user. In the event that the user will not be using this sphygmomanometer in the future and does not wish to register, the user can enter “GUEST.” If GUEST is entered, the measurement results are not stored.

Once the user entry has been completed at ST 3, the cuff pressurizes at a constant rate (ST 4). In a similar manner to FIG. 3, in the inflation process, the CPU 40 extracts the vibrational component (pressure pulse wave amplitude) in conjunction with the volume change of the artery that is superimposed on the pressure change within the air bladder 5A and calculates the blood pressure value and the pulse rate by a prescribed calculation stored in advance, and obtains the estimated blood pressure value (ST 5).

Next, it is determined whether the estimated value obtained at ST 5 is within the reference age (ST 7). In this case, if the user has completed registration, the blood pressure value stored in the memory 7 for storage measured the previous time is used as the reference value. If the user has not completed registration, because there is no measure blood pressure value from a previous time, a value can be used as the reference value that represents hypertension such as 135 mmHg for systolic pressure and 85 mmHg for diastolic pressure as established as a standard for hypertension in home blood pressure by, for example, The Japanese Society of Hypertension.

If it is determined that the estimated value at ST 7 is within the reference range, such fact is displayed on the display unit 4 (ST 8). Thereafter, the air within the cuff is discharged (ST 14), and the measurement is ended. If it is determined that the estimated value at ST 9 exceeds the reference range, the pressurized cuff is gradually decompressed and the blood pressure of the user is measured in the deflation process (ST 10 and 11). The inflation of the cuff in ST 11 does not need to be at a similar inflation rate as that done in ST 5 but can be at a faster rate because there is no need to measure the blood pressure in the inflating process.

The blood pressure calculated at ST 11 is displayed on the display unit 4 at ST 12 together with the display of the person being measured.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. An electronic sphygmomanometer, comprising:
a cuff containing an air bladder for wrapping around a measurement area of a person being examined;
an air charger that inflates the air bladder;
an air discharger that deflates the air bladder;
a sensor that detects changes of pressure within the air bladder during the inflation and/or deflation of the air bladder;
a memory that stores control programs, a reference blood pressure value, and a result of measurement of blood pressure;
a display that displays the result of measurement of blood pressure;
a central processing unit that executes programs stored in the memory and calculating to obtain calculates a blood pressure value of the person being examined based on the changes of pressure within the air bladder, the central processing unit further comprising:
an estimation processing part that calculates an estimated blood pressure value of the person being examined based on the pressure change within the air bladder during the inflation of the air bladder at a first rate; and
a measurement processing part that measures a blood pressure value of the person being examined based on the pressure change within the air bladder during the deflation of the air bladder at a second rate; and
a determination processing part that compares the estimated blood pressure value with the reference blood pressure value and determines whether the estimated blood pressure value is within a predetermined range from the reference blood pressure value,
wherein if the estimated blood pressure value is determined to be within a predetermined range from the reference blood pressure value, measurement operation of the sphygmomanometer is ended without measuring blood pressure by the measurement processing part, and
wherein if the estimated blood pressure value is determined to not be within a predetermined range from the reference blood pressure value, blood pressure of the person being examined is measured by the measurement processing part.

2. The electronic sphygmomanometer according to claim 1, wherein the first rate of inflating the air bladder is faster than the second rate of deflating the air bladder.

3. The electronic sphygmomanometer according to claim 1, further comprising:
an instruction receiving part that receives an instruction to measure blood pressure,
wherein upon receiving an instruction to measure blood pressure by the instruction receiving part, the measurement processing part measures a blood pressure value of the person being examined during the deflation of air bladder without regard to the estimated blood pressure value.

4. The electronic sphygmomanometer according to claim 3, wherein a result of determination processing by the determination processing part is displayed before receiving the instruction by the instruction receiving part.

5. The electronic sphygmomanometer according to claim 1, further comprising:
an instruction receiving part that receives an instruction to measure blood pressure,
wherein the measurement processing part does not measure a blood pressure value of the person being examined during deflation of the air bladder unless the instruction receiving part receives an instruction to measure the blood pressure of the person being examined by the measurement processing part.

6. The electronic sphygmomanometer according to claim 1, further comprising means for entering whether to calculate the estimated blood pressure value by the estimation processing part.

7. The electronic sphygmomanometer according to claim 1, wherein the reference blood pressure value is a systolic pressure and diastolic pressure designated as a standard for high blood pressure for home blood pressure by the Japanese Society of Hypertension.

8. The electronic sphygmomanometer according to claim 1, further comprising means for identifying a user of the sphygmomanometer.

9. The electronic sphygmomanometer according to claim 8, wherein the reference blood pressure value is a blood pressure value stored in the memory as previously measured blood pressure of the same user.

10. An electronic sphygmomanometer, comprising:
a cuff containing an air bladder for wrapping around a measurement area of a person being examined;
a pump that charges air to inflate the air bladder;
a valve that releases air to deflate the air bladder;
a sensor that detects changes of pressure within the air bladder during charging and releasing air to and from the air bladder;
a memory that stores control programs, a reference blood pressure value, and a result of measurement of blood pressure;
a display unit that displays measured blood pressure;
a CPU for executing that executes programs stored in the memory and calculates blood pressure of the person being examined based on the changes of pressure within the air bladder, the CPU further comprising:
an estimation processing part that calculates an estimated blood pressure value of the person being examined based on the pressure change within the air bladder during the inflation of the air bladder at a first rate;
a measurement processing part that measures a blood pressure value of the person being examined based on the pressure change within the air bladder during the deflation of the air bladder at a second rate; and
a determination processing part that compares the estimated blood pressure value obtained by the estimation processing with the reference blood pressure value and determines whether the estimated blood pressure value is within a predetermined range from the referenced blood pressure value,
wherein if the estimated blood pressure value is determined to be within a predetermined range from the reference blood pressure value, the display unit displays the estimated blood pressure value and measurement operation of the sphygmomanometer is ended.

11. The electronic sphygmomanometer according to claim 10, wherein the first rate of inflating the air bladder is faster than the second rate of deflating the air bladder.

12. The electronic sphygmomanometer according to claim 10, wherein if the estimated blood pressure value is determined to not be within a predetermined range from the reference blood pressure value, blood pressure of the person being examined is measured by the measurement processing part, and a result of measurement of blood pressure is displayed by the display unit.

13. The electronic sphygmomanometer according to claim 10, further comprising:
an instruction receiving part that receives instruction whether to quickly check blood pressure,
wherein if the instruction receiving part receives instruction to quickly check blood pressure, blood pressure is measured during the inflation of the air bladder to obtain an estimated blood pressure.

14. The electronic sphygmomanometer according to claim 10, further comprising means for identifying a user of the sphygmomanometer.

15. The electronic sphygmomanometer according to claim 14, wherein the reference blood pressure value is a blood pressure value stored in the memory as previously measured blood pressure of the same user.

16. An electronic sphygmomanometer, comprising:
a cuff containing an air bladder for wrapping around a measurement area of a person being examined;
means for inflating the air bladder;
means for deflating the air bladder;
means for detecting changes of pressure within the air bladder during the inflating and/or deflating the air bladder;
means for storing control programs, a reference blood pressure value, and a result of measurement of blood pressure;
means for displaying measured blood pressure of the person being examined; and
means for executing programs stored in the memory and calculating blood pressure of the person being examined based on the changes of pressure within the air bladder, the means for calculating blood pressure further comprising:
means for calculating an estimated blood pressure value of the person being examined based on the pressure change within the air bladder during the inflation at a first rate; and
means for measuring a blood pressure value of the person being examined based on the pressure change within the air bladder during the deflation at a second rate; and
means for comparing the estimated blood pressure value obtained by the calculating means with the reference blood pressure value,
wherein if the estimated blood pressure value is determined to be within a predetermined range from the reference blood pressure value, measurement operation of the sphygmomanometer is ended without performing measurement by the measuring means, and
wherein if the estimated blood pressure value is determined to be not within a predetermined range from the reference blood pressure value, blood pressure of the person being examined is measured by the measuring means.

17. The electronic sphygmomanometer according to claim 16, wherein the first rate of inflating the air bladder is faster than the second rate of deflating the air bladder.

18. The electronic sphygmomanometer according to claim 16, further comprising means for identifying a user of the sphygmomanometer.

19. The electronic sphygmomanometer according to claim 18, wherein the reference blood pressure value is a blood pressure value stored in the memory as previously measured blood pressure of the same user.

20. A method of monitoring blood pressure by using an electronic sphygmomanometer, comprising:
wrapping a cuff around a measurement area of a person being examined;
inflating an air bladder at a first rate of change;
calculating an estimated blood pressure value of the person being examined based on the pressure change of the air bladder during the inflation of the air bladder;
comparing the estimated blood pressure value with a reference value and determining whether the estimated blood pressure value is within a predetermined range from the referenced blood pressure value,
wherein if the estimated blood pressure value is determined to not be within a predetermined range from the reference value, the air bladder is inflated to a predetermined pressure level at a first rate, the air bladder is deflated to a predetermined pressure level at a second rate, and the change of blood pressure value is measured during the deflation of the air bladder, and
wherein if the estimated blood pressure value measured is determined to be within a predetermined range from the reference blood pressure value, the cuff is deflated without measuring blood pressure.

21. The method of monitoring blood pressure according to claim 20, wherein the first rate of inflating the air bladder is faster than the second rate of deflating the air bladder.

22. The method of monitoring blood pressure according to claim 20, further comprising:
deciding whether to quickly check blood pressure; and
if a user decides to quickly check the blood pressure, measuring the blood pressure of the person being examined during the inflation of the air bladder, and calculating the estimated blood pressure value of the person being examined based on the pressure change of the air bladder during the inflation of the air bladder.
